(19) United States
(12) Patent Application Publication
(10) Pub. No.: US 2023/0028771 A1
(43) Pub. Date: Jan. 26, 2023

(54) DEVICE FOR CONTINUOUSLY PRODUCING AND ANALYZING RNA

(71) Applicant: Academia Sinica, Taipei (TW)

(72) Inventors: James C. LIAO, Taipei (TW); Yi-Chung TUNG, Taipei City (TW)

(73) Assignee: Academia Sinica, Taipei (TW)

(21) Appl. No.: 17/871,896

(22) Filed: Jul. 23, 2022

Related U.S. Application Data
(60) Provisional application No. 63/224,427, filed on Jul. 22, 2021.

(51) Int. Cl. C12P 19/34; C12N 9/12; C12N 9/22; B01L 3/00

(52) U.S. Cl. CPC C12P 19/34; C12N 9/1247; C12N 9/22; B01L 3/502746

(57) ABSTRACT

Disclosed herein is a device for synthesizing ribonucleic acids (RNAs). According to embodiments of the present disclosure, the device comprises an in vitro transcription (IVT) module, a digestion module, and a processor. Optionally, the present device further comprises an IVT reaction monitoring means, a digestion reaction monitoring means, and/or a purifying means. Also disclosed herein are the methods of synthesizing RNA by use of the present device.

### CROSS-REFERENCE TO RELATED APPLICATIONS

This application relates to and claims the benefit of U.S. Provisional Application No. 63/224,427, filed Jul. 22, 2021; the content of the application is incorporated herein by reference in its entirety.

### BACKGROUND OF THE INVENTION

### 1. Field of the Invention

The present disclosure in general relates to the field of nucleic acid synthesis. More particularly, the present disclosure relates to a novel device for synthesizing and analyzing ribonucleic acid (RNA) in a continuous manner.

### 2. Description of Related Art

Messenger RNA (mRNA) vaccine is a new type of vaccine that uses mRNA to stimulate an immune response in the subject thereby protecting the subject against diseases. Specifically, mRNA vaccine consists of an mRNA strand that encodes a disease-specific antigen (e.g., a spike protein of severe acute respiratory syndrome coronavirus 2 (SARS-CoV-2)). Once the mRNA strand is up-taken by immune cells (e.g., dendritic cells), the immune cells use the genetic information of the mRNA strand to produce the antigen. The antigen is then displayed on the cell surface, and serves as an immunogen to stimulate an adaptive immune response against the antigen and the antigen-associated diseases (e.g., COVID-19 caused by SARS-CoV-2).

A major limiting factor of mRNA vaccine in treating diseases is the preparation of mRNA. In general, mRNA may be prepared by solid-phase synthesis or enzymatic transcription. The solid-phase synthesis can only produce RNAs having 40-80 nucleotide in length, in which the yield of RNAs greatly decreases as the length of the nucleotides increases. Regarding the enzymatic transcription, it is typically performed in a batchwise manner. Batchwise production of RNAs is inherently inefficient due to the deadtime between batches or reactions, and contamination risks during sample transportation. Further, the analysis process (i.e., the analysis of RNA amplicon) is separated from the synthesis process that causes unnecessary waste of time and expense.

In view of the foregoing, there exists in the related art a need for a novel device for synthesizing RNA in a more efficient manner.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a device for synthesizing an RNA. The present device comprises an in vitro transcription (IVT) module, a digestion module disposed downstream to the IVT module, and a processor coupled to the IVT module and the digestion module.

In structure, the IVT module comprises,

a first container for housing an RNA polymerase and a deoxyribonucleic acid (DNA) corresponding to the RNA;

a second container for housing ribonucleoside triphosphates (NTPs);

a first mixing unit configured to receive and mix the RNA polymerase, the DNA and the NTPs at a first flow rate to produce a first mixture; and

an IVT chamber configured to perform an IVT reaction with the first mixture from the first mixing unit.

The digestion module comprises,

a third container for housing a deoxyribonuclease (DNase);

a second mixing unit configured to receive and mix the DNase and the IVT reaction product of the IVT chamber at a second flow rate to produce a second mixture; and

a digestion chamber configured to digest the second mixture from the second mixing unit thereby producing the synthesized RNA.

According to embodiments of the preset disclosure, the processor is configured to control the first and second flow rates, and respective conditions (e.g., the temperature and reaction time) of the IVT and the digestion reactions.

According to some embodiments, the first flow rate is about 0.1-100 microliter per minute (μl/min). In these embodiments, the first mixture has a shear stress of about 0.02-20 dyn/cm² under the first flow rate.

According to certain embodiments, the second flow rate is about 0.1-100 μl/min. In these embodiments, the second mixture has a shear stress of about 0.02-20 dyn/cm² under the second flow rate.

Preferably, the device of the present disclosure further comprises an IVT reaction monitoring means, which is coupled to the IVT chamber for monitoring the IVT reaction product in the IVT chamber. Additionally or alternatively, the device of the present disclosure further comprises a digestion reaction monitoring means, which is coupled to the digestion chamber for monitoring the digestion product in the digestion chamber.

Optionally, the present device further comprises a purifying means coupled to the digestion chamber to purify the synthesized RNA.

According to some alternative embodiments, in addition to the IVT module and digestion module, the device of the present disclosure further comprises a monitoring module disposed downstream to the digestion module. In these embodiments, the monitoring module comprises,

a fourth container for housing a dilution buffer;

a third mixing unit configured to receive and mix the dilution buffer and the synthesized RNA of the digestion chamber so as to dilute the synthesized RNA; and

an RNA monitoring means to monitor the diluted product of the third mixing unit.

According to the embodiments, the mixing ratio of the dilution buffer and the synthesized RNA is controlled by the processor.

According to certain exemplary embodiments, the present device further comprises a purifying means that is disposed downstream to the digestion module and is configured to purify the synthesized RNA.

According to some optional embodiments, the present device further comprises a valve that is coupled to the digestion module, the monitoring module and the purifying means, and is configured to control the delivery of the synthetic RNA from the digestion module to the monitoring module or to the purifying means.

Also disclosed herein is a method of synthesizing RNAs by using the device in accordance with any embodiment of the present disclosure. The present device is configured to receive and mix the reactants (i.e., the RNA polymerase, DNA template, and NTPs respectively housed in the first and second containers) in a controlled manner (i.e., at specific flow rates and shear stresses in the first and second mixing units), thereby allowing the reactants to continuously undergo IVT reaction in the IVT chamber and digestion in the digestion chamber, to produce the desired RNA.

According to some embodiments, the first flow rate in the first mixing unit is set as about 0.1-100 μl/min, in which the first mixture has a shear stress of about 0.02-20 dyn/cm² under the first flow rate. According to exemplary embodiments, the IVT reaction in the IVT chamber is carried out at about 16° C. to about 37° C. for at least 1 hour.

According to certain embodiments, the second flow rate in the second mixing unit is set as about 0.1-100 μl/min, in which the second mixture has a shear stress of about 0.02-20 dyn/cm² under the second flow rate. According to exemplary embodiments, the digestion reaction in the digestion chamber is carried out at about 37° C. for at least 10 minutes.

Optionally, the present method further comprises a step of purifying the synthesized RNA by a suitable method, for example, by a purification column.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a schematic diagram depicting a device 10 for synthesizing an RNA according to one embodiment of the present disclosure;

FIGS. 2A to 2C are schematic diagrams respectively depicting devices 20A, 20B, 20C for synthesizing an RNA according to alternative embodiments of the present disclosure;

FIG. 3 is a schematic diagram depicting a device 30 comprising a purifying means according to a further embodiment of the present disclosure;

FIG. 4 is a schematic diagram depicting a device 40 comprising a monitoring module according to one embodiment of the present disclosure;

FIG. 5 is a schematic diagram depicting a device 50 comprising a monitoring module and a purifying means according to another embodiment of the present disclosure; and

FIG. 6 is a line chart depicting the concentration of RNA prepared by the present device or batch production according to Example 1 of the present disclosure.

In accordance with common practice, the various described features/elements are not drawn to scale but instead are drawn to best illustrate specific features/elements relevant to the present invention. Also, like reference numerals and designations in the various drawings are used to indicate like elements/parts.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “flow rate” refers to a volume of a fluid per unit time. As used herein, the term “flow rate” refers to the rate of passage of a fluid through a point in an unit (e.g., the first or second mixing unit of the present disclosure) for the fluid over a unit of time. For instance, a typical unit measurement of “flow rate” is liter or microliter per minute. The term “flow rate” may refers to an actual flow rate, a measured flow rate, or an estimated flow rate.

The term “shear stress” refers to the ratio of force to area. As used herein, the term “shear stress” refers to the mechanical stress on the molecules (e.g., DNA, RNA and/or protein) dissolved in the solution caused by the fluidic velocity of a flow in an unit (e.g., the first or second mixing unit of the present disclosure). The “shear stress” is usually measured at a constant flow rate through a conduit or unit having a constant diameter and/or length.

The term “valve” as used herein refers to any flow regulating device or system. For example, the term “valve” can include, without limitation, any device or system that controllably allows, prevents, or inhibits the flow of the air or fluid through a passageway (e.g., the monitoring module and purifying means of the present device). The term “valve” can be a direction valve, pinch valve, rotary valve, stop cock, pressure valve, shuttle valve, mechanical valve, electrical valve, electro-mechanical flow regulator, or the combination thereof. According to one exemplary embodiment, the valve is a 6-way valve or the like usually used for high-performance liquid chromatography (HPLC).

### II. Description of the Invention

The present invention is directed to a device for synthesizing and analyzing RNA in a continuous manner. Compared to conventional batchwise production of RNA (i.e., manufacturing RNA as specified groups or amounts within a time frame), the present device is advantageous at least in three points: (1) minimizing the deadtime between reactions and/or batches; (2) reducing the contamination risks during sample transportation; and (3) providing a real-time monitoring, evaluation and control of the synthesized RNA.

The first aspect of the present disclosure is thus directed to a device, which is characterized by incorporating two modules, i.e., an IVT module and a digestion module disposed downstream to the IVT module. According to embodiments of the present disclosure, the IVT module is configured to mix transcription materials (e.g., DNA template and NTPs) and reagents (e.g., RNA polymerase) at a suitable flow rate and shear stress to perform an IVT reaction. The digestion module is configured to digest the DNA template in the IVT reaction product by mixing the IVT reaction product with DNase thereby producing desired RNA amplicons.

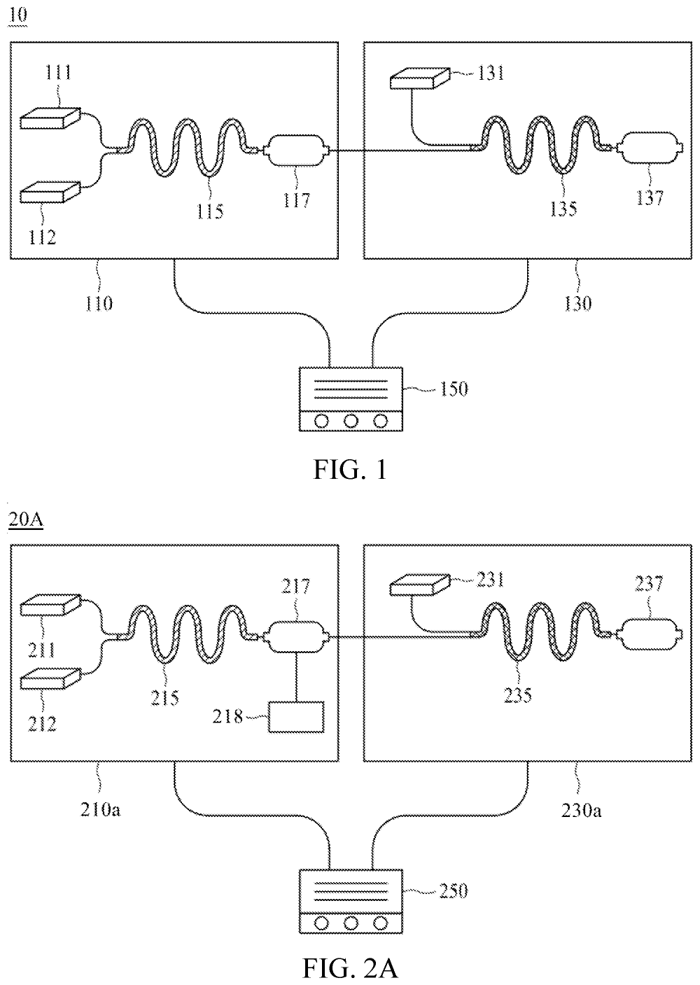

Reference is made to FIG. 1, which depicts a device 10 for synthesizing an RNA. The device 10 includes at least, an IVT module 110, a digestion module 130 disposed downstream to the IVT module 110, and a processor 150 coupled to both modules for controlling and monitoring the IVT module 110 and the digestion module 130.

The IVT module 110, as its name implies, is where the IVT reaction occurs and comprises in its structure, a first container 111 that houses RNA polymerase and DNA template (i.e., DNA molecule serving as a template for the synthesis of a complementary RNA transcript); a second container 112 that houses NTPs (including ATP, UTP, CTP and GTP); a first mixing unit 115 that is coupled to the first and second containers 111, 112, and is configured to respectively receive and mix the compounds supplied from the first and second containers 111, 112 at a first flow rate so as to produce a first mixture (i.e., the mixture of the RNA polymerase, DNA template and NTPs); and an IVT chamber 117 that is disposed downstream to the first mixing unit 115 and is configured to perform an IVT reaction with the first mixture supplied from the first mixing unit 115.

In practice, the RNA polymerase, DNA template and NTPs are respectively dissolved in a suitable buffer (e.g., Tris or Tris-HCl buffer) and stored in the first and second containers 111, 112. Depending on intended purposes, the RNA polymerase may be SP6 RNA polymerase, T7 RNA polymerase, T3 RNA polymerase, or any other RNA polymerase known to synthesize RNA amplicon from DNA template. For the purpose of optimizing the IVT reaction, the buffer may optionally contain an RNase inhibitor, metal ion (e.g., Mg²⁺), dithiothreitol (DTT) and/or TRITON™ X-100.

Then, the RNA polymerase, DNA template and NTPs in the first container 111 and the second container 112 are respectively delivered to and mixed in the first mixing unit 115 at a stable flow rate. According to some embodiments of the present disclosure, the flow rate is about 0.01-1,000 μl/min (e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950 or 1,000 μl/min) that results in a shear stress of about 0.002-200 dyn/cm² (e.g., 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190 or 200 dyn/cm²). Preferably, the flow rate is about 0.1-100 μl/min that results in a shear stress of about 0.02-20 dyn/cm². According to certain embodiments, the flow rate and shear stress in the first mixing unit 115 ensure that the RNA polymerase, DNA template and NTPs can be effectively mixed without molecule-molecule dissociation.

To synthesize the RNA amplicon from the DNA template, the mixture of the RNA polymerase, DNA template and NTPs is then delivered from the first mixing unit 115 to the IVT chamber 117, where the IVT reaction is conducted. According to certain embodiments, the IVT reaction is carried out at about 16° C. to about 37° C. (e.g., about 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C. or 37° C.) for at least 1 hour (e.g., 1, 1.5, 2, 2.5, 3, 3.5, 4, 4.5, 5, 5.5, 6, 6.5, 7, 7.5, 8, 8.5, 9, 9.5 or 10 hours, or longer); preferably, the IVT reaction is carried out at about 37° C. for 1-3 hours. According to one working example, the IVT reaction is carried out at 37° C. for 3 hours. As could be appreciated, the conditions (e.g., the temperature and reaction time) would vary with the factors, such as the length of synthesized RNA, the type of RNA polymerase, and the concentration of reacting agents. A skilled artisan may select suitable procedure for performing the IVT reaction in accordance with intended purposes.

The thus-produced IVT reaction product contains both the DNA template and the synthesized RNA amplicon. To remove the DNA template, the IVT reaction product is subjected to a digestion treatment in the digestion module 130. The digestion module 130 comprises in its structure, a third container 131 that houses a DNase; a second mixing unit 135 that is coupled to the IVT chamber 117 and the third container 131, and is configured to respectively receive and mix the DNase and the IVT reaction product at a second flow rate to produce a second mixture (i.e., a mixture of the IVT reaction product and DNase); and a digestion chamber 137 that is coupled to the second mixing unit 135 and is configured to digest the second mixture supplied from the second mixing unit 135 thereby producing the synthesized RNA.

According to some embodiments of the present disclosure, the DNase (i.e., an endonuclease digesting single- and double-stranded DNA) housed in the third container 131 is dissolved in a suitable buffer. Examples of buffer suitable for dissolving the DNase include, but not limited to, phosphate buffered saline (PBS), diethyl pyrocarbonate (DEPC)-treated water, Tris buffer, Tris-HCl buffer or the combination thereof. Optionally, the buffer may contain one or more metal ion, such as Mg²⁺, Ca²⁺ and/or Mn²⁺ to optimize the digestion efficacy.

The IVT reaction product supplied from the IVT chamber 117 and the DNase housed in the third container 131 are then mixed in the second mixing unit 135 at a stable flow rate and shear stress so as to ensure the IVT reaction product and DNase are effectively mixed without molecule-molecule dissociation. According to some embodiments of the present disclosure, the flow rate is about 0.01-1,000 μl/min that results in a shear stress of about 0.002-200 dyn/cm². Preferably, the flow rate is about 0.1-100 μl/min that results in a shear stress of about 0.02-20 dyn/cm².

The well-mixed mixture of the IVT reaction product and DNase leaving the second mixing unit 135 are then subjected to DNase digestion in the digestion chamber 137. According to certain embodiments of the present disclosure, the digestion reaction is carried out at about 37° C. for at least 10 minutes (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59 minutes; or 1, 2, 3, 4, 5 or 6 hours, or longer). In some exemplary embodiments, the incubation at 37° C. for 15 minutes is sufficient to digest and remove the DNA template from the IVT reaction product. According to one working example, the digestion reaction is carried out at 37° C. for 1 hour. A skilled artisan may select suitable procedure for performing the digestion reaction in accordance with intended purposes.

Both the IVT module 110 and the digestion module 130 are under the control of the processor 150. According to embodiments of the present disclosure, the processor 150 is configured to control the operations of the IVT module 110 and the digestion module 130, including the first flow rate in the first mixing unit 115, the second flow rate in the second mixing unit 135, the IVT reaction conditions (e.g., the temperature and reaction time) in the IVT chamber 117, and the digestion reaction conditions (e.g., the temperature and reaction time) in the digestion chamber 137.

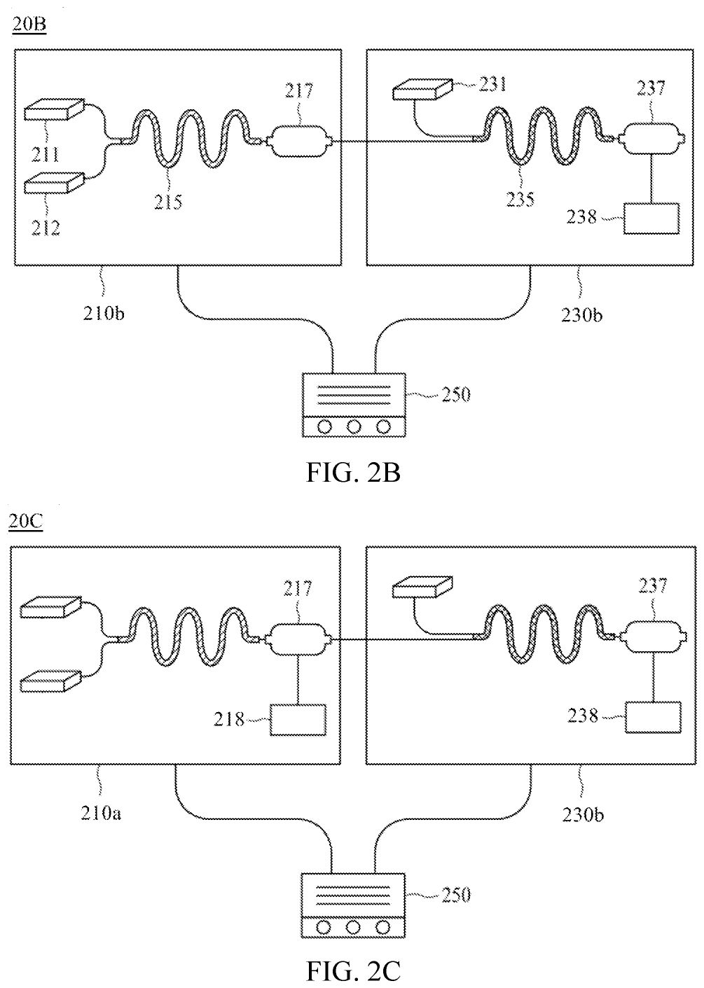

Optionally, the present device may further comprise a monitoring means to detect and/or analyze the IVT reaction product and/or the digestion product in a real-time manner. Reference is made to FIG. 2A, which depicts a device 20A for synthesizing RNA. The device 20A differs from that in FIG. 1 (i.e., the device 10) in that it further includes an IVT reaction monitoring means 218 coupled to the IVT chamber 217. The components and their arrangements in the IVT module 210a (including the first and second containers 211, 212, the first mixing unit 215, and the IVT chamber 217) and the digestion module 230a (including the third container 231, the second mixing unit 235, and the digestion chamber 237) are similar to that of the device 10. The detailed descriptions of these technical features are thus omitted for the sake of brevity.

As noted above, the IVT module 210a differs from that in FIG. 1 (i.e., the IVT module 110) in that it further includes an IVT reaction monitoring means 218 coupled to the IVT chamber 217. After receiving the IVT reaction product from the IVT chamber 217, the IVT reaction monitoring means 218 provides a real-time analysis of the IVT reaction product so that a skilled artisan may decide whether to continue the digestion reaction, and/or to adjust the conditions of the IVT reaction in time, e.g., promptly adjust the concentration and/or ratio of the transcription materials/reagents in the first and second containers 211, 212, the flow rate and the shear stress in the first mixing unit 215, and/or the temperature and/or reaction time in the IVT chamber 217.

Alternatively, the device of the present disclosure further comprises a monitoring means to detect and/or analyze the digestion product. Reference is made to FIG. 2B, which depicts a device 20B for synthesizing RNA. The digestion module 230b differs from that in FIG. 1 (i.e., the digestion module 130) in that it further includes a digestion reaction monitoring means 238 coupled to the digestion chamber 237 so as to provide a real-time monitoring to the digestion product in the digestion chamber 237. The components and their arrangements in the IVT module 210b (including the first and second containers 211, 212, the first mixing unit 215, and the IVT chamber 217) and the digestion module 230b (including the third container 231, the second mixing unit 235, and the digestion chamber 237) are similar to that of the device 10. The detailed descriptions of these technical features are thus omitted for the sake of brevity. After receiving the digestion product from the digestion chamber 237, the digestion reaction monitoring means 238 provides a real-time analysis of the digestion product so that a skilled artisan may adjust the condition of the digestion reaction in time, e.g., promptly adjust the DNase concentration in the third container 231, the mixing ratio of the digestion product from the IVT chamber 217 and the DNase from the third container 231, the flow rate and the shear stress in the second mixing unit 235, and/or the temperature and/or reaction time in the digestion chamber 237.

Still alternatively, the present device may incorporate both the IVT reaction monitoring means and the digestion reaction monitoring means. Reference is made to FIG. 2C, which depicts a device 20C for synthesizing RNA. Compared to the device 10, the device 20C is characterized by having an IVT reaction monitoring means 218 coupled to the IVT chamber 217, and a digestion reaction monitoring means 238 coupled to the digestion chamber 237. In this way, the device 20C is useful in monitoring the IVT reaction product in the IVT chamber 217 and the digestion product in the digestion chamber 237 in a real-time manner, and providing a feedback control to the processor 250 so as to optimize the IVT and digestion reactions.

Depending on desired purposes, each of the IVT reaction monitoring means 218 and the digestion reaction monitoring means 238 may independently be a ultraviolet (UV) spectroscopy (also known as “UV-visible spectrophotometry” that monitors the RNA amplicon via measuring its UV absorbance at a wavelength of 260 nm), a surface plasmon resonance (SPR)-based sensor (e.g., BIACORE™ system that monitors RNA transcription by measuring RNA polymerase dissociation and transcript release rate at elongation and termination positions), a fluorescence or chemiluminescence detector (e.g., capillary electrophoresis or microfluidic chip that identifies RNA molecule by using its complementary nucleic acid probe conjugated with a fluorescent or chemiluminescent molecule), or any devices or apparatuses known to detect, monitor and/or quantify RNA molecules, for example, mass spectrometry (MS), liquid chromatography (LC; e.g., HPLC or fast-performance liquid chromatography (FPLC)), or the combination thereof. Alternatively, each of the IVT reaction monitoring means 218 and the digestion reaction monitoring means 238 may independently be a device or apparatus comprising one or more sensors for measuring the properties (e.g., physical, optical and electrochemical properties) of the IVT reaction product and/or digestion product; for example, each of the IVT reaction monitoring means 218 and the digestion reaction monitoring means 238 may independently comprise one or more fluidic sensors for monitoring the pressure, flow rate, bubble and/or pH value of the IVT reaction product and/or digestion product; one or more optical sensors for monitoring the light intensity, scattering light and/or absorption spectrum of the IVT reaction product and/or digestion product; and/or one or more conductivity sensors for monitoring the conductivity and/or resistivity of the IVT reaction product and/or digestion product.

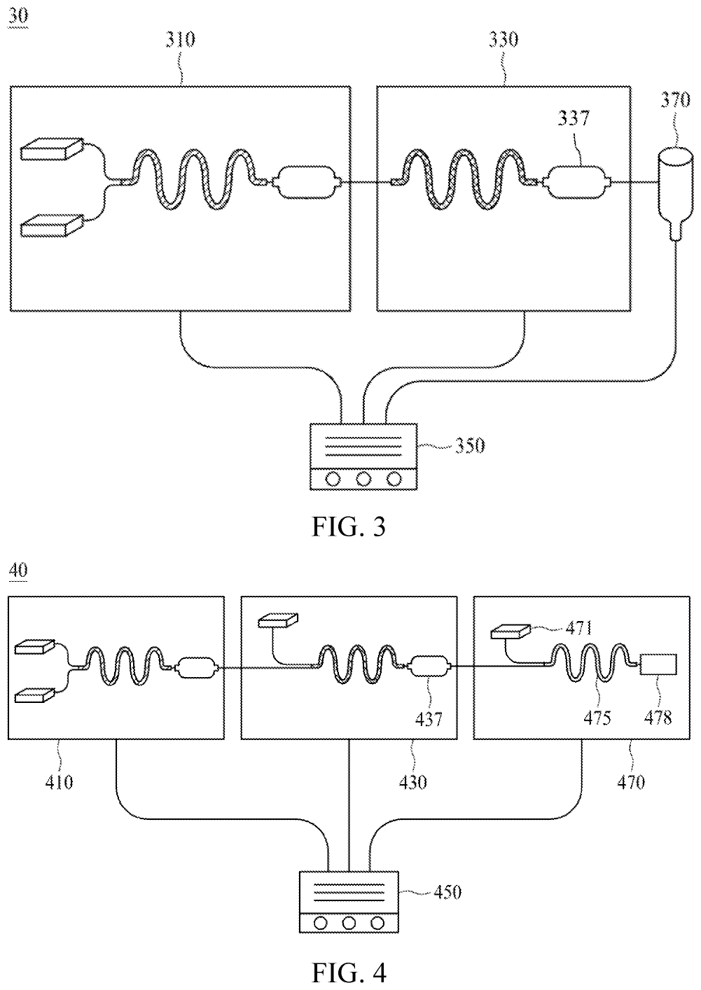

Optionally, in addition to the IVT module, digestion module and processor, the present device may further include a purifying means coupled to the digestion module to purify the digestion product, i.e., the synthesized RNA. Reference is made to FIG. 3, in which the device 30 further includes a purifying means 370 (e.g., a column) disposed downstream to the digestion chamber 337 for isolating and/or purifying the thus synthesized RNA. According to some embodiments of the present disclosure, the operation of the purifying means 370 is controlled by the processor 350. Examples of the purifying means suitable for use in the present disclosure include, but are not limited to, purification column, magnetic bead, filter, electrophoresis and the combination thereof.

FIG. 4 depicts a device 40 according to alternative embodiments of the present disclosure. The device 40 has a structure similar to the device 10 of FIG. 1, except that in addition to the IVT module 410 and digestion module 430, the device 40 further comprises a monitoring module 470 that is coupled to the processor 450 and is disposed downstream to the digestion module 430. The monitoring module 470 comprises in its structure, a fourth container 471 for housing a dilution buffer; a third mixing unit 475 that is coupled to the fourth container 471 and the digestion chamber 437, and is configured to receive and mix the dilution buffer and the digestion product of the digestion chamber 437 so as to dilute the synthesized RNA; and an RNA monitoring means 478 to detect and/or analyze the diluted product (i.e., the diluted RNA) in a real-time manner.

According to some embodiments, the mixing ratio of the dilution buffer and the synthesized RNA, the flow rate in the third mixing unit 475, and the operation of the RNA monitoring means 478 are controlled by the processor 450.

As described above, the RNA monitoring means 478 may be an UV spectroscopy, SPR-based sensor, fluorescence or chemiluminescence detector, or any devices or apparatuses known to detect, monitor and/or quantify RNA molecules, for example, MS, HPLC, FPLC, or the combination thereof Alternatively, the RNA monitoring means 478 may be a device or apparatus comprising one or more sensors for measuring the properties (e.g., physical, optical and electrochemical properties) of the diluted RNA; for example, the RNA monitoring means 478 may comprise one or more fluidic sensors for monitoring the pressure, flow rate, bubble and/or pH value of the diluted RNA; one or more optical sensors for monitoring the light intensity, scattering light and/or absorption spectrum of the diluted RNA; and/or one or more conductivity sensors for monitoring the conductivity and/or resistivity of the diluted RNA.

As could be appreciated, the dilution buffer housed in the fourth container 471 would vary with the type of the RNA monitoring means 478; for example, in the case when the RNA monitoring means is an UV spectroscopy, the dilution buffer is preferably RNase-free water; in the case when the RNA monitoring means is a SPR-based sensor, then the dilution buffer may be a phosphate buffer, HEPES buffer, sodium acetate buffer, acetate buffer, Tris(hydroxymethyl)aminomethane (Tris)-HCl buffer, or the combination thereof in the case when the RNA monitoring means is a fluorescence detector, then the dilution buffer may be RNase-free water, HEPES buffer, or Tris-HCl buffer. A skilled artisan may choose suitable dilution buffers in accordance with intended purpose.

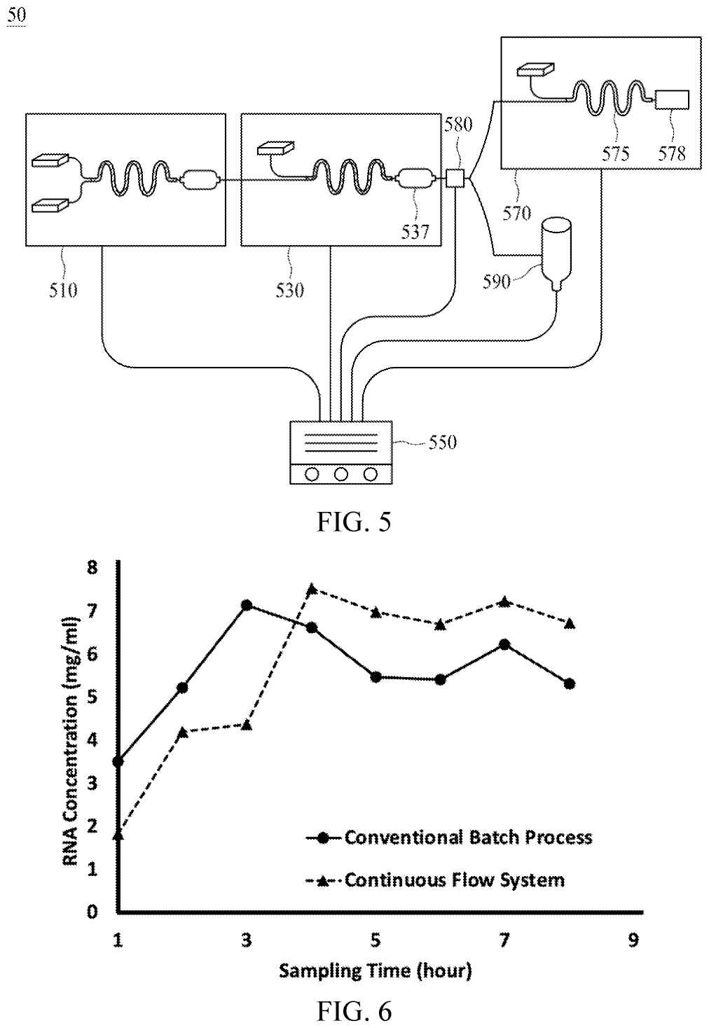

Optionally, in addition to the IVT module, digestion module and monitoring module as depicted in FIG. 4, the present device may further include a purifying means (e.g., purification column, magnetic bead, filter, electrophoresis, or the combination thereof) coupled to the digestion module so as to purify the digestion product, i.e., the synthesized RNA. Reference is made to FIG. 5, which depicts a device 50 according to alternative embodiments of the present disclosure. The device 50 has a structure similar to the device 40 of FIG. 4, except that in addition to the IVT module 510, the digestion module 530 and the monitoring module 570, the device 50 further includes a purifying means 590 and a valve 580 operably coupled to the processor 550. As depicted in FIG. 5, the monitoring module 570 and the purifying means 590 are disposed downstream and coupled to the digestion module 530 via the valve 580 (e.g., a 6-way valve). The valve 580 is configured to control the flow direction of the digestion product from the digestion chamber 537 to the third mixing unit 575 or the purifying means 590. In practice, the digestion product is first delivered from the digestion chamber 537 to the third mixing unit 575 followed by detecting and/or analyzing by the RNA monitoring means 578 as described above. Once the amount and/or quality of the synthesized RNA determined by the monitoring means 578 are/is equal to or higher than a predetermined level, the flow direction of the digestion product is switched from the third mixing unit 575 to the purifying means 590 via the valve 580 so as to ensure the quality and quantity of the purified RNA.

The device of the present disclosure provides advantages of,

(1) continuous operation to avoid deadtime between reactions/batches;

(2) controlled shear stress and stable laminar flow for effective mixing in gentle conditions and minimizing protein-nucleic acid dissociation;

(3) precise control of reaction time;

(4) real-time in situ control of RNA growth;

(5) real-time quality check and possible feedback control to minimize reagent cost;

(6) being compatible with column purification;

(7) high controllability and being amenable to automation so as to reduce the error or variation caused by human operation;

(8) being scale-friendly and good manufacturing practice (GMP)-compliance friendly; and

(9) being capable of establishing large scale reaction directly from the reaction conditions in small scale.

The second aspect of the present disclosure pertains to a method of synthesizing RNA by use of the present device in accordance with any embodiments mention above. The present device is configured to receive and mix the reactants (i.e., the RNA polymerase, DNA template, and NTPs respectively housed in the first and second containers) in a controlled manner (i.e., at specific flow rates and shear stresses in the first and second mixing units), thereby allowing the reactants to continuously undergo IVT reaction in the IVT chamber and digestion in the digestion chamber, to produce the desired RNA.

Preferably, the RNA polymerase, DNA template and NTPs are respectively dissolved in a suitable buffer (e.g., Tris or Tris-HCl buffer), followed by subjecting to the first and second containers. The buffer may optionally comprise an RNase inhibitor, metal ion (e.g., Mg²⁺), dithiothreitol (DTT) and/or TRITON™ X-100 to optimize the IVT reaction.

According to some embodiments of the present disclosure, the flow rate of the reactants in the first mixing unit is set as about 0.01-1,000 μl/min, which results in a shear stress of about 0.002-200 dyn/cm² in the first mixture (i.e., the mixture of the RNA polymerase, DNA template and NTPs). Preferably, the flow rate of the reactants in the first mixing unit is set as about 0.1-100 μl/min, which results in a shear stress of about 0.02-20 dyn/cm² in the first mixture. According to some preferred embodiments, the IVT reaction in the IVT chamber is carried out at about 16° C. to about 37° C. for at least 1 hour.

According to certain embodiments of the present disclosure, the flow rate of the reactant in the second mixing unit is set as about 0.01-1,000 μl/min, which results in a shear stress of about 0.002-200 dyn/cm² in the second mixture (i.e., the mixture of the IVT reaction product and DNase). Preferably, the flow rate of the reactants in the second mixing unit is set as about 0.1-100 μl/min, which results in a shear stress of about 0.02-20 dyn/cm² in the second mixture. According to some preferred embodiments, the digestion reaction in the digestion chamber is carried out at about 37° C. for at least 10 minutes.

Optionally, the method further comprises the step of purifying the synthesized RNA. The technique for isolating and purifying RNA molecules is known by a skilled artisan, for example, column, phenol-chloroform extraction or magnetic bead.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

The proof-of-concept experiments were performed using the reagents included in a commercially available RNA synthesis kit with a customized DNA template. In the IVT module, the raw materials were fed into the system from two separate inlets with each flow rate of 3.6 μl/hr (7.2 μl/hr in total) and reacted at 37° C. for 3 hours in a continuous flow manner. The reacted IVT crude was then introduced into the DNase module and mixed with DNase with a flow rate of 28.8 μl/hr and reacted at 37° C. for 1 hour. The reacted solution was then diluted using the buffer in a dilution module in the volumetric ratio of 1:30. The flow rates with 35 times higher values were also tested in the system and the results were confirmed to yield similar system performance.

### Example 1 Characterizing RNA Synthesized by the Present Device

The IVT reaction product obtained by the present device was analyzed by a fluorescence-based quantification system, tape station, and electrophoresis. The data of FIG. 6 indicated that the device could stably produce RNA after the 3-hour reaction with comparable quantity as the conventional batch process. The RNA length was confirmed using the tape station (data not shown), and the optical and electrical conductance can also be in-situ monitored to ensure the stable process operation (data not shown). The electrophoresis results demonstrated that the DNA can be successfully digested in the DNase module (data not shown). The results also confirmed that the present device can be continuously working for at least 8 hours with stable production capacity.

It will be understood that the above description of embodiments is given by way of example only and that various modifications may be made by those with ordinary skill in the art. The above specification provides a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A device for synthesizing a ribonucleic acid (RNA), comprising,
an in vitro transcription (IVT) module comprising,
a first container for housing an RNA polymerase and a deoxyribonucleic acid (DNA) corresponding to the RNA;
a second container for housing ribonucleoside triphosphates (NTPs);
a first mixing unit configured to receive and mix the RNA polymerase, the DNA and the NTPs at a first flow rate to produce a first mixture; and
an IVT chamber configured to perform an IVT reaction with the first mixture from the first mixing unit;
a digestion module that is disposed downstream to the IVT module and comprises,
a third container for housing a deoxyribonuclease (DNase);
a second mixing unit configured to receive and mix the DNase and the IVT reaction product of the IVT chamber at a second flow rate to produce a second mixture; and
a digestion chamber configured to digest the second mixture from the second mixing unit thereby producing the synthesized RNA; and
a processor coupled to the IVT module and the digestion module, for controlling the first and second flow rates, and respective conditions of the IVT and the digestion reactions.

2. The device of claim 1, wherein the first flow rate is about 0.1-100 μl/min.

3. The device of claim 2, wherein the first mixture has a shear stress of about 0.02-20 dyn/cm² under the first flow rate.

4. The device of claim 1, wherein the second flow rate is about 0.1-100 μl/min.

5. The device of claim 4, wherein the second mixture has a shear stress of about 0.02-20 dyn/cm² under the second flow rate.

6. The device of claim 1, further comprising an IVT reaction monitoring means coupled to the IVT chamber for monitoring the IVT reaction product in the IVT chamber.

7. The device of claim 1, further comprises a digestion reaction monitoring means coupled to the digestion chamber for monitoring the digestion product in the digestion chamber.

8. The device of claim 1, further comprising a purifying means coupled to the digestion chamber to purify the synthesized RNA.

9. The device of claim 1, further comprises a monitoring module that is disposed downstream to the digestion module and comprises,
a fourth container for housing a dilution buffer;
a third mixing unit configured to receive and mix the dilution buffer and the synthesized RNA of the digestion chamber so as to dilute the synthesized RNA; and
an RNA monitoring means to monitor the diluted product of the third mixing unit,
wherein the mixing ratio of the dilution buffer and the synthesized RNA is controlled by the processor.

10. The device of claim 9, further comprising a purifying means that is disposed downstream to the digestion module to purify the synthesized RNA.

11. The device of claim 10, further comprising a valve that is coupled to the digestion module, the monitoring module and the purifying means, and is configured to control the delivery of the synthetic RNA from the digestion module to the monitoring module or the purifying means.

12. A method of synthesizing a ribonucleic acid (RNA) by using a device, wherein the device comprises,
an in vitro transcription (IVT) module comprising,
a first container for housing an RNA polymerase and a deoxyribonucleic acid (DNA) corresponding to the RNA;
a second container for housing ribonucleoside triphosphates (NTPs);
a first mixing unit configured to receive and mix the RNA polymerase, the DNA and the NTPs at a first flow rate to produce a first mixture; and
an IVT chamber configured to perform an IVT reaction with the first mixture from the first mixing unit;
a digestion module that is disposed downstream to the IVT module and comprises,
a third container for housing a deoxyribonuclease (DNase);
a second mixing unit configured to receive and mix the DNase and the IVT reaction product of the IVT chamber at a second flow rate to produce a second mixture; and
a digestion chamber configured to digest the second mixture from the second mixing unit thereby producing the synthesized RNA; and
a processor coupled to the IVT module and the digestion module, for controlling the first and second flow rates, and respective conditions of the IVT and the digestion reactions;
wherein the method comprises respectively providing the RNA polymerase and the DNA corresponding to the RNA to the first container, and providing the NTPs to the second container, so as to synthesize the RNA.

13. The method of claim 12, wherein the first flow rate is set as about 0.1-100 μl/min.

14. The method of claim 13, wherein the first mixture has a shear stress of about 0.02-20 dyn/cm² under the first flow rate.

15. The method of claim 12, wherein the IVT reaction is carried out at about 16° C. to about 37° C. for at least 1 hour.

16. The method of claim 12, wherein the second flow rate is set as about 0.1-100 μl/min.

17. The method of claim 16, wherein the second mixture has a shear stress of about 0.02-20 dyn/cm² under the second flow rate.

18. The method of claim 12, wherein the digestion reaction is carried out at about 37° C. for at least 10 minutes.

19. The method of claim 12, further comprising a step of purifying the synthesized RNA.
